(19) United States
(12) Patent Application Publication
(10) Pub. No.: US 2026/0221232 A1
(43) Pub. Date: Jul. 30, 2026

(54) SPATIALLY INFORMED CLUSTERING, INTEGRATION, AND DECONVOLUTION OF SPATIAL TRANSCRIPTOMICS WITH GRAPHST

(71) Applicant: Agency for Science, Technology and Research, Singapore (SG)

(72) Inventors: Yahui LONG, Singapore (SG); Jinmiao CHEN, Singapore (SG)

(21) Appl. No.: 19/149,063

(22) PCT Filed: Jan. 4, 2024
(86) PCT No.: PCT/SG2024/050008

§ 371 Date: Jul. 17, 2025

(30) Foreign Application Priority Data
Jan. 18, 2023 (SG) 10202300148U

(51) Int. Cl. G16B 40/30; G16B 25/10

(52) U.S. Cl. CPC G16B 40/30; G16B 25/10

(57) ABSTRACT

Systems and methods for processing spatial transcriptomics data and generating insights relating cell and tissue states. The system and methods include spatial clustering, integration of data from multiple tissue samples and integration of scRNA-sequence data and spatial transcriptomics data. The systems and methods incorporate graph self-supervised contrastive learning and graph neural networks to perform spatial transcriptomics data processing.

### TECHNICAL FIELD

This disclosure generally relates to methods and systems for processing spatial transcriptomics data. In particular, the disclosure provides methods and systems for spatial clustering, spatial transcriptomics data integration, and single cell RNA sequencing transfer onto spatial transcriptomics.

### BACKGROUND

This background description is provided for the purpose of generally presenting the context of the disclosure. Contents of this background section are neither expressly nor impliedly admitted as prior art against the present disclosure.

The human body has an extensive range of cells that move between states during life such as development, disease, and regeneration. Despite coming from the same zygote, the types and states of the cell are significantly influenced by both internal processes and environmental factors.

Complex heterogeneities in the cellular architecture are seen during the progression of the cell through the proliferation and differentiation states to produce diverse cell types for organ formation. The ability of the same cell types to perform different roles is made possible by the cellular heterogeneity that exists between various tissues in terms of appearance, function, and gene expression profiles. Linking gene expression of cells with their spatial distribution is crucial for understanding the tissue's emergent properties and pathology. Current spatial transcriptomics (ST) combine gene profiling and spatial information to yield greater insights into both healthy and diseased tissues. Spatial information is also useful for inferring cell-cell communications.

Advances in spatial transcriptomics technologies have facilitated the characterization of gene expression profiles at single-cell resolution while retaining information about the spatial tissue context. However, conventional methods of analysis of transcriptomics data often suffer from limitations in accurately deciphering tissue gene expression patterns in a tissue, systematically analyzing multiple tissue slices, and accurately integrating single-cell RNA-seq (scRNA-seq) and spatial transcriptomics (ST) data. The conventional methods employ unsupervised learning and they often show sub-optimal clustering performance as the boundaries of identified domains are often fragmented and poorly match pathological annotations. As ST ground-truth segmentation is usually not available, supervised learning cannot be employed to improve performance. Further, most current analysis methods are suitable for only a single tissue slice and cannot jointly identify spatial domains from multiple tissue slices. Current technological limitations also prevent ST from achieving single cell resolution with gene coverage comparable to single cell RNA sequencing (scRNA-seq).

It would be desirable to overcome or ameliorate at least one of the above-described problems, or at least to provide a useful alternative.

### SUMMARY

In one embodiment, the present disclosure provides a system for transcriptome data processing, the system comprising a memory and one or more processor(s), the memory comprising program code executable by the processor(s) to:

receive a first plurality of records of spatial gene expression data of a first tissue sample, wherein the records comprise gene expression data and location data indicating a location of the gene expression data in the first tissue sample;
select a first subset of the first plurality records corresponding to spatial gene expression data of variable genes;
transform the records in the selected first subset into a graph data structure based on the location data;
process the graph data structure using a neural network implementing contrastive self-supervised learning to obtain latent gene expression data, wherein the latent gene expression data is a dimensionally reduced representation of the first subset of the records; and
transforming the latent gene expression data into reconstructed gene expression data using a decoder neural network,
wherein records in the reconstructed gene expression data are associated with location data in the received spatial gene expression data.

Some embodiments relate to a system for transcriptome data processing, the system comprising a memory and one or more processor(s), the memory comprising program code executable by the processor(s) to:
receive a first plurality of records of single cell RNA sequence data relating to a plurality of cells from a tissue sample;
receive a second plurality of records (Hs) of spatial gene expression data of the tissue sample, wherein the records comprise spatial gene expression data and location data indicating a location of the spatial gene expression data in the tissue sample;
process the first plurality of records using a self-supervised Auto-Encoder neural network to generate cell representation records (HC);
project the cell representation records (HC) on the second plurality of records using an untrained mapping matrix (M′) to obtain predicted spatial gene expression records (Hs′);
process the predicted spatial gene expression records using a neural network implementing contrastive learning to obtain reconstructed spatial gene expression records (Hs); and train mapping matrix (M′) to generate a trained mapping matrix (M) based on the reconstructed spatial gene expression records (Hs) and the cell representation records (HC).

Some embodiments relate to a method for transcriptome data processing, the method comprising:
receiving a first plurality of records of spatial gene expression data of a first tissue sample, wherein the records comprise gene expression data and location data indicating a location of the gene expression data in the first tissue sample;
selecting a first subset of the first plurality records corresponding to spatial gene expression data of variable genes;
constructing a graph data structure based on the location data; and
processing the graph data structure and the selected first subset using a deep learning framework implementing graph self-supervised contrastive learning to obtain latent gene expression data;
wherein the latent gene expression data is a dimensionally reduced representation of the first subset of the records.

The method of some embodiments further comprises processing using a clustering model the reconstructed gene expression data to identify a plurality of clusters in the first tissue sample, wherein each cluster comprises locations with similar gene expression profiles.

The method of some embodiments, further comprises:
receiving a second plurality of records of spatial gene expression data of a second tissue sample, wherein the second plurality of records comprise gene expression data and location data indicating a location of the gene expression data in the second tissue sample;
aligning the location data of the first and second plurality of records into a common coordinate space; and
selecting a second subset of the second plurality records corresponding to spatial gene expression data of variable genes,
wherein the graph data structure is obtained by based on the location data originating from the first and second tissue samples; and the latent gene expression data comprises data originating from both the first and second tissue samples.

The method of some embodiments further comprises transforming the latent gene expression data into reconstructed gene expression data using a decoder neural network, wherein records in the reconstructed gene expression data are associated with locations in both the first and second tissue samples.

The method of some embodiments further comprises processing using a clustering model the reconstructed gene expression data to identify a plurality of clusters in the first and second tissue samples, wherein each cluster comprises locations with similar gene expression profiles.

Some embodiments relate to a method for transcriptome data processing, the method comprising:
receiving a first plurality of records of single cell RNA sequence data relating to a plurality of cells from a tissue sample;
receiving a second plurality of records (Hs) of spatial gene expression data of the tissue sample, wherein the records comprise spatial gene expression data and location data indicating a location of the spatial gene expression data in the tissue sample;
processing the first plurality of records using a self-supervised Auto-Encoder neural network to generate cell representation records (HC);
projecting the cell representation records (HC) on the second plurality of records using an untrained mapping matrix (M′) to obtain a predicted spatial gene expression records (Hs′);
processing the spatial gene expression records using a deep learning framework implementing graph self-supervised contrastive learning to obtain reconstructed spatial gene expression records (Hs); and training the untrained mapping matrix (M′) to generate a trained mapping matrix (M) based on the reconstructed spatial gene expression records (Hs) and the cell representation records (HC).

The method of some embodiments further comprises projecting the cell representation records (HC) on the second plurality of records using the trained the mapping matrix (M) to perform spatial localization of cell types in the tissue sample.

### BRIEF DESCRIPTION OF THE DRAWING

Some embodiments of systems and methods for processing transcriptomics data, in accordance with the present disclosure, will now be described, by way of non-limiting example only, with reference to the accompanying drawing in which:

FIGS. 1 and 2 illustrate methods for transcriptome data processing.

FIG. 3 illustrates an overview of GraphST for spatial clustering, integration and deconvolution of spatial transcriptomics.

FIG. 4 illustrates a system for implementing the methods of FIGS. 1 and 2, or the framework of FIG. 3.

### DETAILED DESCRIPTION

The systems and methods of the disclosure may also be referred to as GraphST or GraphST model. GraphST comprises a versatile graph contrastive self-supervised learning framework that incorporates spatial location information and gene expression profiles for spatial clustering, integration and transfer of spatial transcriptomics. With simple usability and fast computing capability, GraphST may enable distinguishing heterogeneity within spatial transcriptomics data and provide insights into cellular states within each tissue.

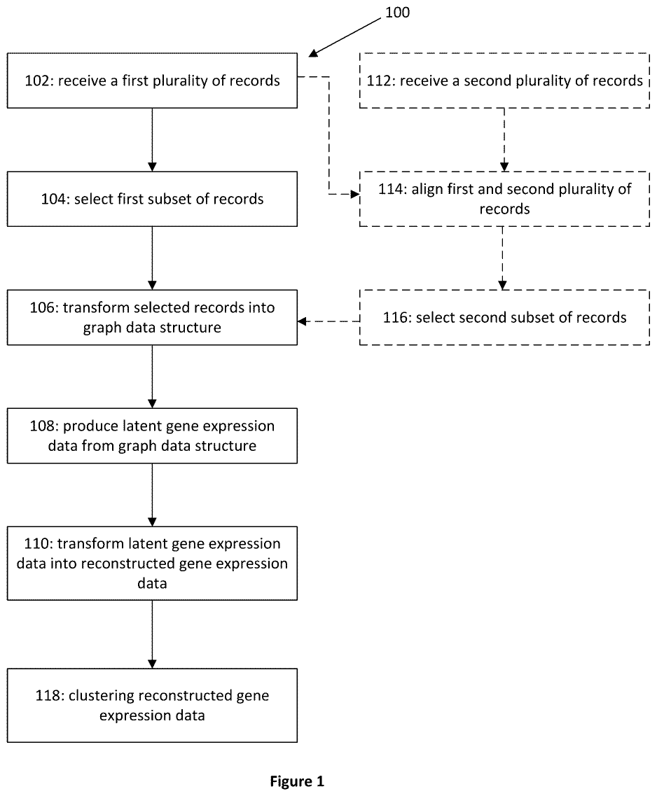

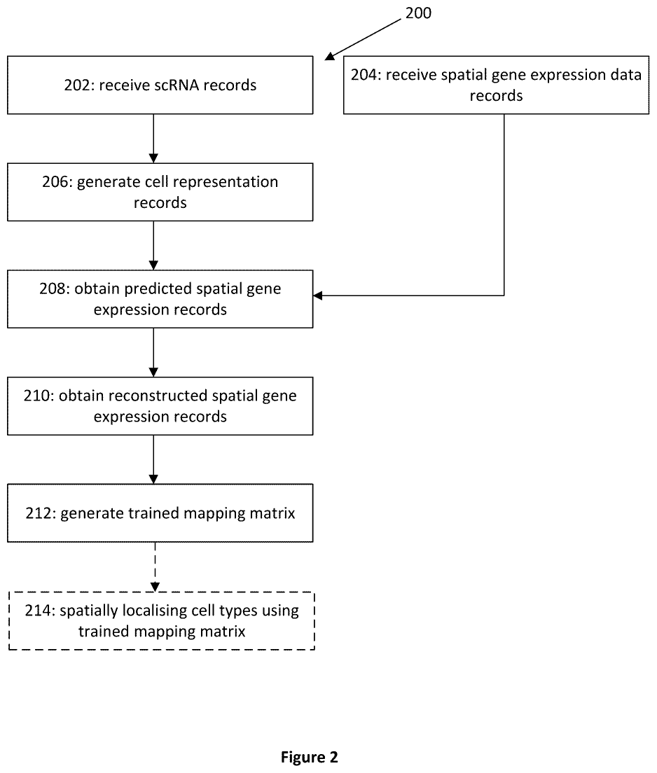

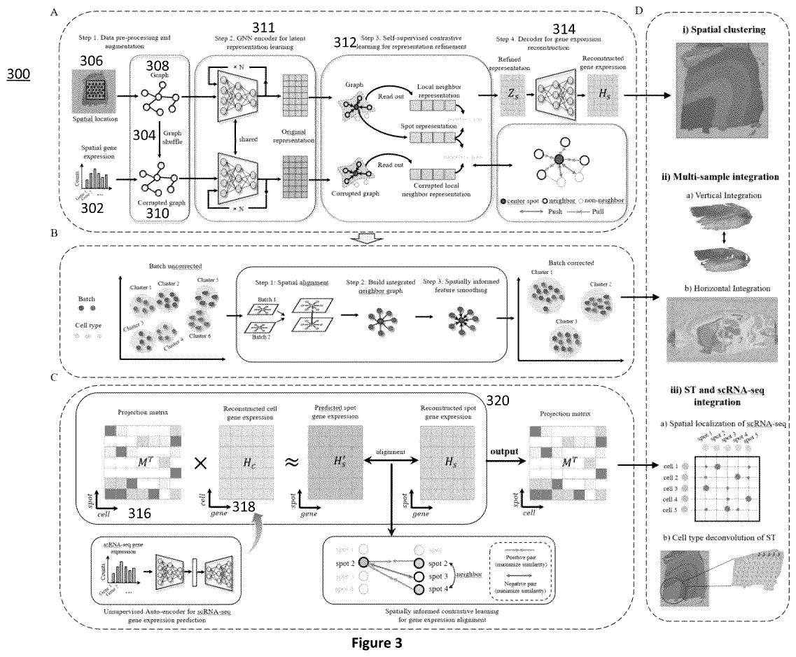

Some methods used herein are embodied by FIG. 1, in which the method 100 for transcriptome data processing involves receiving a plurality of records (step 102), selecting a subset of those records (104), transforming the records in the selected subset into a graph data structure (step 106) that can be processed to produce latent gene expression data (step 108). The latent gene expression data produced by step 108 is a dimensionally reduced representation of the subset of the original records received at step 102. Latent gene expression data is data obtained at 320 of (FIG. 3, image (C)) during the transformation of gene expression data by a neural network implementing contrastive self-supervised learning. The latent gene expression data may represent a dimensionally reduced representation that enables the processing of the gene expression data more feasible by conventional computer systems.

Step 102 can involve receiving records in any desired manner. The records may come for a single data source or database, or may be produce by integrating datasets from multiple sources.

The records received at step 102 comprise spatial gene expression data of a first tissue sample. The records also comprise location data indicating a location of the gene expression data in the first tissue sample.

### Transcriptomics Data Examples

For the task of spatial segmentation, the GraphST model implemented, at least in part, by method 100 takes (e.g. receives) the first plurality of records of spatial gene expression data per step 102. The records may correspond to samples taken from one or more spatial gene expression datasets. In the example illustrated here, the samples are taken from four datasets. The spatial gene expression data are of a first tissue sample, and the records comprise the gene expression data and location data indicating a location of the gene expression data in the first tissue sample.

The datasets used for this purpose may be generated from the Visium platform or on any other platform. The publicly derived datasets used herein are summarized as follows: (1) the first dataset was the Lieber Institute for Brain Development (LIBD) human dorsolateral prefrontal cortex (DLPFC) data, which included twelve slices from three individuals. The number of spots for each slice range from 3,460 to 4,789. (2) The second dataset on human breast cancer tumor samples contained 3,798 spots. (3) The third dataset was mouse breast cancer derived from non-treated metastatic tumors and includes 1,950 spots. (4) The fourth dataset comprised of mouse brain anterior including 2,695 spots from the publicly available 10X Genomics Data Repository.

In some embodiments, data in the first plurality of records must be integrated. Integration can comprise performing one or both of vertical and horizontal integration. For the task of multiple sample integration in experiments, the GraphST methods were executed on two samples from 10X Visium platform for vertical and horizontal integration, respectively.

For vertical integration, the analysis covered mouse breast cancer tissue. For the data being integrated during vertical integration, each spatial gene expression data point may be vertically adjacent one or more other spatial gene expression data points, and these vertically adjacent data points may be integrated. In experiments, vertical integration involved using two sets of samples derived from this tissue, of which each sample was composed of two vertically adjacent sections. The numbers of spots ranged from 1,868 to 3,042 for each section.

For horizontal integration, GraphST was implemented to analyze spatial transcriptomic mouse brain (anterior & posterior) tissue. The mouse brain anterior and posterior were horizontally split. Both anterior and posterior contain two sections (section 1 and 2) and the number of spots for each section are about 3,000. All of the sections were downloaded from the 10X Genomics Data Repository.

At step 104, a subset of the records received at step 102 is selected. That subset is selected to correspond to spatial gene expression data of highly variable genes (HVGs). The HVGs are selected using SCANPY function ‘sc.pp.highly_variable_genes( )’ where the HVGs are identified by calculating a normalized variance for each gene. First, the data are standardized (i.e. z-score normalization per feature) with a regularized standard deviation. Next, the normalized variance is computed as the variance of each gene after the transformation. The genes are ranked by the normalized variance. Genes with variance above a predetermined threshold variance (e.g. predetermined by experimentation as those to which the present methods apply) or top N genes may then be specified as highly variable.

Once the variable genes are identified, the subset of records selected at step 104 is transformed into a graph data structure at step 106, that transformation being based on the location data in the subset of records.

Step 108 can then be performed using a neural network that implements self-supervised learning. Processing the graph data structure at step 108, using the neural network, results in latent gene expression data being obtained. This latent gene expression data is a dimensionally reduced representation of the original records from the selected subset.

A further method 200 described herein can be used for transcriptome data processing for hybrid datasets such as single cell RNA (scRNA) sequence data and spatial gene expression data. The method 200 comprises receiving records comprising scRNA sequence data relating to a plurality of cells from a tissue sample (step 202), and records (Hₛ) of spatial gene expression data of the tissue sample. As with step 202, the spatial gene expression data includes location data indicating a location of the spatial gene expression data in the tissue sample.

Step 206 of method 200 involves processing the records received at step 204, using a self-supervised Auto-Encoder neural network to generate cell representation records (H_{C}). Encoder neural network comprises a neural network that transforms a higher dimensional dataset into a lower dimensional dataset while conserving the information embedded in the dataset. These cell representation records (H_{C}) can then be projected on the records received at step 202, using an untrained mapping matrix (M′)—step 208. This projection obtains predicted spatial gene expression records (Hₛ′). The resulting records Hs' can then be processed using a neural network implementing contrastive learning to obtain reconstructed spatial gene expression records (Hₛ)—step 210. Reconstructed gene expression data is gene expression data that is more information-dense and/or discriminative to more accurately and/or efficiently support subsequent analysis for tasks such as spatial segmentation.

Now having the reconstructed records Hs and the cell representation records H_{C}, the mapping matrix M′ can be trained to produce a trained mapping matrix M—step 212. The trained mapping matrix can then be used to map new cell representation records to spatial gene expression records and, conversely, to map new spatial gene expression records to cell representation records.

For the task of scRNA-seq and ST integration in experiments, GraphST was tested on three sets of human or mouse datasets. The first dataset, received at step 202, was from a mouse brain anterior sample. The spatial data contains 2695 spots and 32,285 genes. scRNA-seq data are from mouse whole cortex and hippocampus 10× website, which profiled>1.1 million cells and 22,764 genes from multiple cortical areas and the hippocampal formation. The second dataset, received at step 204, was from a human breast cancer sample. The scRNA-seq data comprised a dataset of 46,080 cells with 5,000 gene expressions according to cell type information. GraphST adopted data obtained from human breast cancer tissue with 3,798 spots and 36,601 genes as ST data. The final dataset originated from a human brain sample. The ST data in some experiments included data of 3,639 spots and 33,538 genes. The single-cell data comprised snRNA-seq data profiled on archived post-mortem dorsolateral prefrontal cortex (BA9) tissue using the 10X Genomics Chromium platform. From this dataset, some experiments involved randomly sampling 78,886 cells with 30,062 genes.

The above-noted of sources of data are merely exemplary. GraphST provides flexible techniques for processing ST data and scRNA data to distinguish heterogeneity within spatial transcriptomics data and provide insights into cellular states within tissues irrespective of the origin of the data or the source/method used to obtain the relevant data.

### Data Pre-Processing

For all ST data, pre-processing involved first filtering out spots without annotations (if the dataset has annotations). Then gene expression counts in the records received at step 204 (and 102) can be normalized to library size. For example, raw gene expression counts may be log-transformed and normalized to library size via SCANPY package. For steps 104 and 116, this step being capable of also being performed in the method 200 for one or both of the ST and scRNA records, the top 3,000 highly variable genes (HVGs) were treated as input features of the GraphST model—the number of genes and the number of records can be selected depending on preference, application or other factors. Similarly, for scRNA-seq data, raw gene expression counts were first log-transformed and normalized to library size and then we chose the top 3,000 highly variable genes as cell input features of the model. To implement the task of scRNA-seq and ST data integration, the pre-processed HVGs of scRNA-seq and ST data were further aligned and the overlapped genes were used as the final input features of both spots and cells.

### Spatial Clustering

FIG. 3 illustrates the method 100 in the context of a deep learning framework 300 for spatial clustering of transcriptomics data. Given a spatial transcriptomics dataset per step 102, part (A) of FIG. 3 discloses data pre-preprocessing of the gene expression data 302 correlating genes and counts, and spatial location data 306 indicating a location of the gene expression data in the tissue sample. Data pre-processing and augmentation involves generating corrupted graph 310 corresponding to original graph 308 as per step 106. The corrupted graph 310 is generated through data augmentation with original graph as input. Data augmentation is achieved by randomly swapping or shuffling 304 features between nodes (i.e., spots) in the original graph.

An encoder 311 comprising a graph neural network (GNN) can be used to analyse and extract features from the original and corrupted graphs 308, 310 for latent representation learning and producing original representation of latent gene expression data as per step 108.

Next, a graph contrastive self-supervised learning framework 312 refines the original representation by pairing local neighbor representation and spot representation as positive pair, and pairing spot representation and corrupted local neighbor representation as negative pair.

Subsequently, a decoder for gene expression reconstruction 314 transforms the refined representation of latent gene expression data into reconstructed gene expression data—i.e. the decoder 314 reverses the representations Zₛ back into original space to reconstruct gene expression Hₛ, per step 110. A decoder neural network comprises a neural network that transforms a lower dimensional dataset into a higher dimensional dataset, wherein the higher dimensional dataset corresponds to a domain of interest. In this disclosure, the domain of interest is gene expression data associated with locations or spots. This reverse transformation associates records in the reconstructed gene expression data with location data in the received spatial gene expression data.

The learning process can be guided by loss minimization—for example, by minimizing both self-reconstruction loss and contrastive loss. Self-reconstruction loss enforces representations Hₛ to completely reserve gene expression features and spatial location information. Contrastive loss makes full use of data itself to make representations Hₛ more discriminative and informative. Per step 110, after model training, with the output Hₛ, some embodiments incorporated a non-spatial assignment algorithm, such as mclust, to cluster spots into different spatial domains. Each cluster could be regarded as a spatial domain, which contains spots with similar gene expression profiles and tight spatial locations.

Embodiments may implement deep learning frameworks of an alternative structure to perform the described methods. Deep learning frameworks may comprise neural networks implementation computational logic to process information.

As shown in part (B) of FIG. 3, reconstructed gene expression data Hₛ obtained in part (A) is then subjected to spatial clustering. Existing ‘mclust’ clustering algorithm is utilized to segment spots into different spatial domains. ‘mclust’ is a contributed R package for model-based clustering, classification, and density estimation based on finite normal mixture modelling. It provides functions for parameter estimation via the EM algorithm for normal mixture models with a variety of covariance structures, and functions for simulation from these models. Also included are functions that combine model-based hierarchical clustering, EM for mixture estimation and the Bayesian Information Criterion (BIC) in comprehensive strategies for clustering, density estimation and discriminant analysis. Additional functionalities are available for displaying and visualizing fitted models along with clustering, classification, and density estimation results.

### Vertical and Horizontal Integration of Multiple ST Data

In some embodiments, the GraphST model performs integration analysis of multiple ST data. Taking ST data obtained from two tissues (steps 102 and 112) as an example, before the input to the model, embodiments may first align the images per step 114—e.g. the location data of the first and second plurality of records, received at steps 102 and 112, respectively, may be aligned into a common coordinate space. An algorithm for performing this alignment may be the PASTE algorithm that is used to align Hematoxylin-Eosin (H&E) stained images. If two tissue slices are from different regions in the same tissue, such as the mouse brain anterior and posterior, the alignment is beneficial to ensure that the same domains across two tissue slices are horizontally continuous. If two tissue slices are from the same regions, such as mouse brain anterior section 1 and section 2, the alignment is required to maximize the overlapped areas between the two sections. With the aligned data (including spatial location and gene expressions) as inputs, GraphST can generate spot representations for both ST data (FIG. 3, image (A)), which can be used for joint spatial clustering. For the transformation step 106 onwards, a subset of the aligned second set of records can be selected based on those records representing variable genes.

scRNA-Seq and ST Data Integration

Some embodiments comprise a spatially informed contrastive learning module for ST and scRNA-seq data integration (FIG. 3, image (C)), implementing method 200. This may be performed by adopting a self-supervised Auto-Encoder to learn cell representation H_{c} from scRNA-seq gene expression, per step 206. At 316, with the spot and cell representations Hₛ and H_{c}, embodiments aim to project scRNA-seq data into spatial transcriptomes via a trainable mapping matrix M (which may be referred to as untrained mapping matrix M′ and trained mapping matrix M-bearing in mind that M may be further trained), denoting the probability that cells are projected into each spot of spatial data, to produced predicted spatial gene expression records (Hₛ′). This process is implemented per step 210, and at 318, by aligning the predicted spatial gene expression records

$${H}_{s}^{′}$$

to the reconstructed spatial gene expression Hₛ via a contrastive learning mechanism, where the similarities of positive pairs (i.e., spot i and its neighbors) are maximized while those of negative pairs (i.e., spot i and its non-neighbors) are minimized. After model training, mapping matrix M can be trained, per step 212. In some embodiments, mapping matrix M can then be applied for projecting scRNA-seq data into spatial transcriptomes per step 214.

Variable genes or spatially variable genes comprise genes whose expression distributions display significant dependence on their spatial locations. Such genes contribute strongly to cell-to-cell variation within a homogenous cell population—the term “highly variable genes” and “variable genes” will be used interchangeably herein, unless context dictates otherwise.

Location data comprises data identifying a spot in a tissue sample that was the source of a record of gene expression data. The location data may comprise three-dimensional coordinates or other such representations of location within a tissue sample.

Graph data structures comprise non-linear data structures made up of a finite number of nodes or vertices and the edges that connect them. The graph data structures of the embodiments associate particular locations or spots with a node and rely on the location data to define the vertices or edges. The graph data structures of the embodiments model the location of the gene expression data within the tissue samples.

Contrastive self-supervised learning comprises computational techniques that generally aim to learn to compare records in a domain through an objective function exemplified below:

$$ℒ = {\text{?}}_{x , {x}^{+} , {x}^{-}} [ - log ( \frac{{e}^{{f ( x )}^{T} f ( {x}^{+} )}}{{e}^{{f ( x )}^{T} f ( {x}^{+} )} + {e}^{{f ( x )}^{T} f ( {x}^{-} )}} ]$$

where x+ is similar to x, x− is dissimilar to x and f is an encoder (representation function). The similarity measure in the context of this disclosure relates to the similarity in gene expression data across the records.

FIG. 3 (D) shows downstream analysis tasks. GraphST enables three main tasks, including spatial clustering, multi-sample integration, and ST and scRNA-seq integration

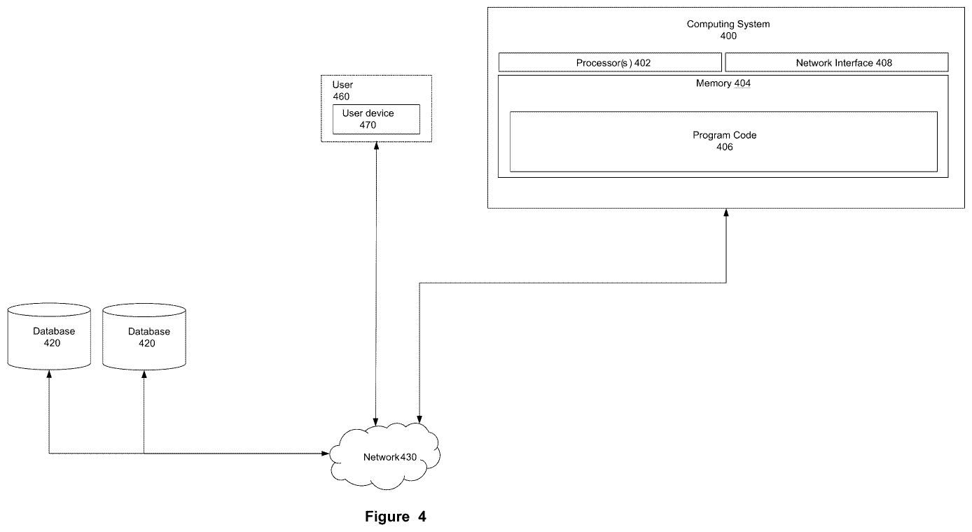

FIG. 4 illustrates a block diagram of a system for transcriptome data processing. According to FIG. 4, the system comprises at least one processor 402, memory 404 accessible to the processor 402 and a network interface 408 to facilitate communication with a plurality of databases 420 and a user computer device 470 of a user 460. Program code 406 provided in memory 404 comprises instructions executable by the processor 402 to perform at least a part of the method of the embodiments described herein. Notably, while individual computer systems are described in FIG. 4, any such computer system may be distributed across multiple servers or multiple devices, or some functionality may be consolidated into a single server or device, without departing from the purposive intent of the present disclosure.

The users device 470 can facilitate extraction of data (e.g. ST or scRNA data) from databases 420, and initiation of method 100, 200, or implementation of framework 300, by processor(s) 402 of computing system 400. The users device 470 may comprise a query engine that can be used to input a query comprising, for example, a new ST record for mapping to a cell representation record (e.g. to generate a record based on the new ST record, or to produce that record then cross-reference it against cell representation records to identify the record corresponding to the new ST record). The user computing device 470 may be a personal or handheld computing device such as a smartphone or a tablet. Network 430 facilitates communication between the various devices (e.g. user device 470 and databases 420) and may include one or more communication networks including the internet, cell phone networks etc.

One or more database 420 are also accessible to the system 400. Each database 420 may comprises records such as one or more of ST records and/or scRNA records, mapping matrices or neural network models used in framework 300.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1. A system for transcriptome data processing, the system comprising a memory and one or more processors, the memory comprising program code executable by the one or more processors to:
receive a first plurality of records of spatial gene expression data of a first tissue sample, wherein the records comprise gene expression data and location data indicating a location of the gene expression data in the first tissue sample;
select a first subset of the first plurality records corresponding to spatial gene expression data of variable genes;
transform the records in the selected first subset into a graph data structure based on the location data;
process the graph data structure using a neural network implementing contrastive self-supervised learning to obtain latent gene expression data; and
transform the latent gene expression data into reconstructed gene expression data using a decoder neural network,
wherein the latent gene expression data is a dimensionally reduced representation of the first subset of the records; and
wherein records in the reconstructed gene expression data are associated with location data in the received spatial gene expression data.

2. A system for transcriptome data processing, the system comprising a memory and one or more processors, the memory comprising program code executable by the one or more processors to:
receive a first plurality of records of single cell RNA sequence data relating to a plurality of cells from a tissue sample;
receive a second plurality of records (Hₛ) of spatial gene expression data of the tissue sample, wherein the records comprise spatial gene expression data and location data indicating a location of the spatial gene expression data in the tissue sample;
process the first plurality of records using a self-supervised Auto-Encoder neural network to generate cell representation records (H_{C});
project the cell representation records (H_{C}) on the second plurality of records using an untrained mapping matrix (M′) to obtain predicted spatial gene expression records (Hₛ′);
process the predicted spatial gene expression records using a neural network implementing contrastive learning to obtain reconstructed spatial gene expression records (Hₛ); and
train mapping matrix (M′) to generate a trained mapping matrix (M) based on the reconstructed spatial gene expression records (Hₛ) and the cell representation records (H_{C}).

3. A method for transcriptome data processing, the method comprising:
receiving a first plurality of records of spatial gene expression data of a first tissue sample, wherein the records comprise gene expression data and location data indicating a location of the gene expression data in the first tissue sample;
selecting a first subset of the first plurality records corresponding to spatial gene expression data of variable genes;
constructing a graph data structure based on the location data; and
processing the graph data structure and the selected first subset using a deep learning framework implementing graph self-supervised contrastive learning to obtain latent gene expression data;
wherein the latent gene expression data is a dimensionally reduced representation of the first subset of the records.

4. The method as claimed in claim 13, further comprising:
transforming the latent gene expression data into reconstructed gene expression data using a decoder neural network; and
processing, using a clustering model, the reconstructed gene expression data to identify a plurality of clusters in the first tissue sample, wherein each cluster comprises locations with similar gene expression profiles.

5. The method as claimed in claim 3, further comprising:
receiving a second plurality of records of spatial gene expression data of a second tissue sample, wherein the second plurality of records comprise gene expression data and location data indicating a location of the gene expression data in the second tissue sample;
aligning the location data of the first and second plurality of records into a common coordinate space; and
selecting a second subset of the second plurality records corresponding to spatial gene expression data of variable genes,
wherein the graph data structure is obtained based on the location data originating from the first and second tissue samples; and
the latent gene expression data comprises data originating from both the first and second tissue samples.

6. The method as claimed in claim 5, further comprising:
transforming the latent gene expression data into reconstructed gene expression data using a decoder neural network,
wherein records in the reconstructed gene expression data are associated with locations in both the first and second tissue samples.

7. The method as claimed in claim 6, further comprising processing using a clustering model the reconstructed gene expression data to identify a plurality of clusters in the first and second tissue samples, wherein each cluster comprises locations with similar gene expression profiles.

8. A method for transcriptome data processing, the method comprising:
receiving a first plurality of records of single cell RNA sequence data relating to a plurality of cells from a tissue sample;
receiving a second plurality of records (Hₛ) of spatial gene expression data of the tissue sample, wherein the records comprise spatial gene expression data and location data indicating a location of the spatial gene expression data in the tissue sample;
processing the first plurality of records using a self-supervised Auto-Encoder neural network to generate cell representation records (H_{C});
projecting the cell representation records (H_{C}) on the second plurality of records using an untrained mapping matrix (M′) to obtain a predicted spatial gene expression records (Hₛ′);
processing the spatial gene expression records using a deep learning framework implementing graph self-supervised contrastive learning to obtain reconstructed spatial gene expression records (Hₛ); and
training the untrained mapping matrix (M′) to generate a trained mapping matrix (M) based on the reconstructed spatial gene expression records (Hₛ) and the cell representation records (H_{C}).

9. The method as claimed in claim 8, wherein the method further comprises:
projecting the cell representation records (H_{C}) on the second plurality of records using the trained the mapping matrix (M) to perform spatial localization of cell types in the tissue sample.
